(19) United States
(12) Patent Application Publication
(10) Pub. No.: US 2025/0003987 A1
(43) Pub. Date: Jan. 2, 2025

(54) BIOMARKERS OF HIDDEN OBESITY FOR USE IN PREVENTATIVE HEALTHCARE

(71) Applicant: Randox Laboratories Ltd., Crumlin, Co Antrim (GB)

(72) Inventors: Paul Innocenzi, Crumlin Co Antrim (GB); Stephen Peter Fitzgerald, Crumlin Co Antrim (GB); Ivan McConnell, Crumlin Co Antrim (GB)

(21) Appl. No.: 18/707,545

(22) PCT Filed: Nov. 8, 2022
(86) PCT No.: PCT/GB2022/052824

§ 371 Date: May 3, 2024

(30) Foreign Application Priority Data
Nov. 9, 2021 (GB) 2116070.0

(51) Int. Cl. G01N 33/68; G01N 33/74

(52) U.S. Cl. CPC G01N 33/6893; G01N 33/74; G01N 2333/4716; G01N 2800/04

(57) ABSTRACT

A method is described for the identification of individuals with a BMI of less than 25 who have a biochemical profile that corresponds to an overweight or obese phenotype. The method of highlighting this hidden obesity makes use of various biomarker combinations that can incorporate various analytes including leptin and complement C3.

### BACKGROUND OF THE INVENTION

The covid 19 viral pandemic has highlighted a different pandemic that has been ongoing for decades and which continues to escalate, that of overweight and obesity (OvOb). The major risk factors for hospitalisation, severe illness and death for covid-infected individuals are age and obesity/overweight. Major health institutions world-wide such as the NHS, the CDC and WHO use the body mass index (BMI) as an indicator of OvOb and it is calculated using the height and weight measurements of an individual in which <18.5 is underweight, 18.5-<25.0 is healthy, 25.0-<30.0 is overweight and ≥30.0 is obese. OvOb indicates excessive fat mass and is considered both a disease and, more commonly, as a risk factor of disease including atherosclerosis, insulin resistance and type 2 diabetes. It is thought that adipose tissue dysfunction, possibly due to increased adipocyte number/size, may lead to insulin resistance and ultimately type 2 diabetes. The mechanistic relationship between obesity and a poor covid 19 disease outcome is uncertain and may be related to endocrinal dysfunction in adipose tissue resulting in an imbalance between pro-inflammatory and anti-inflammatory mediating compounds leading to inflammation and a deleterious exaggerated immune response. A BMI measurement of 25 or greater as well as one of 30 or greater enables the identification of at-risk patients of both a poor covid infection outcome and of other chronic inflammatory conditions. Although BMI is an effective screening tool it does not directly measure fat-not everyone with a BMI of 25 or greater will develop disease and not everyone below a BMI of 25 will be free of disease. Furthermore, body fat presents as subcutaneous adipose tissue (SAT) residing beneath the skin and as visceral adipose tissue (VAT) which accumulates in the abdominal cavity and can envelop internal organs, and each is thought to present different disease risk factors. VAT is considered to be more metabolically active and is associated with low grade inflammation and the secretion of pro-inflammatory mediators which is thought to increase disease severity in several conditions including covid patients (e.g. Kaess B. M. et al. 2012, Diabetologia, 55:2622-2630; Watanabe M. et al. 2020, Metabolism Clinical and Experimental, 11:154319) and has been associated with the so-called ‘skinny-fat’ body phenotype, a disease risk indicator. The BMI metric contains no information on either the anatomical location or the qualitative characteristics of the fat and furthermore does not discriminate between fat and muscle tissue. In addition, the ratio of the SAT to VAT volume can vary considerably between individuals, including those with the same BMI. The direct measurement of fat volume (proxy measurement for fat mass) in its form as subcutaneous adipose tissue and visceral adipose tissue using imaging techniques such as MRI, CAT scans and ultrasound offers a more robust fat measurement, but these techniques lack fat qualitative information, require specialist and experienced operators e.g. radiologists, and are expensive.

Therefore, improved methods of BMI obesity/overweight classification in relation to disease risk are required.

### SUMMARY OF THE INVENTION

An approach to disease risk classification in the context of BMI obesity/overweight classification using biochemical measurements is described. It has been found that the standard method of identifying overweight and obesity by using BMI is susceptible to overlooking individuals who have a BMI of less than 25.00 but who possess an overweight/obese biochemical profile. Biomarkers for use in the invention include complement C3, leptin, ferritin, hs-CRP, triglycerides, small LDL, LDL, complement C4, insulin and C-peptide most of which are common to both males and females. Identification of this overweight/obese biochemical phenotype through novel combinations of biomarkers in individuals with a BMI<25.00 enables a practical and relatively inexpensive approach to the diagnosis of this hidden pre-disease state which can lead to pathological conditions such as insulin resistance, diabetes and atherosclerosis, supporting a preventative healthcare approach.

According to a first aspect of the invention, there is a method of identifying an individual with an overweight/obese biochemical phenotype, said individual having a BMI of less than 25.00, comprising measuring the concentration of leptin or at least two biomarkers from an ex vivo sample obtained from the individual, and based on the measurement(s) establishing whether the individual has an overweight/obese phenotype, wherein the at least two biomarkers are chosen from complement C3, leptin, triglycerides, hs-CRP, C-peptide, small LDL, LDL, ferritin, insulin and complement C4.

According to a second aspect of the invention, there is an assay comprising the steps of:
conducting on a sample of blood, plasma, serum or urine from a subject an analysis of leptin or an analysis of any of the biomarker combinations defined in any of claims 1, 2 and 7 to 9;
providing a measurement of the concentration of said biomarker(s), wherein said subject has a BMI of less than 25.00, and wherein an increase in the measured concentration of said biomarker(s) compared to a control sample or value is indicative of overweight/obese phenotype.

According to a third aspect of the invention, there is method of detecting biomarkers of an overweight/obese phenotype in a patient having a BMI of less than 25.00, comprising
measuring the level of leptin or two or more of the biomarkers as defined in any of claims 1, 2 and 7 to 9, in a sample or samples obtained from said patient, and
determining whether the measured level for each biomarker exceeds a control value for each biomarker.

According to a fourth aspect of the invention, there is a kit for use in the diagnosis of an overweight/obese phenotype in a subject comprising analyte-specific probes for at least two of the biomarkers defined in any of claims 1, 2 and 7 to 9.

According to a fifth aspect of the invention, there is a diagnostic test kit incorporating at least two different binding ligands, each specific to a single biomarker, the at least two biomarkers comprising complement C3 and leptin or leptin and small LDL.

According to a sixth aspect of the invention there is the use of complement C3 and leptin or leptin and small LDL as biomarkers of an individual with an overweight or obese biochemical phenotype.

### DESCRIPTION OF THE DRAWINGS

The invention is described with reference to the accompanying drawings, in which:

FIG. 1 shows a correlation matrix of all healthy males showing Spearman r-value for BMI and biomarker pairs. Age was not significantly related to BMI.

FIG. 2 shows a correlation matrix of all healthy females showing Spearman r-value for BMI and biomarker pairs. Age was not significantly related to BMI.

FIG. 3 shows a graph relating to multiple logistic regression analysis using leptin+complement C3+triglycerides biomarkers to delineate BMI ≥25.00 and BMI<25.00 in males with no underlying health conditions. The median Visceral Adipose Tissue (VAT) ratings represents a visceral fat score for four cohorts grouped according to BMI (<25.00 and ≥25.00) and predicted probability cut-off value shown by the dotted line (<0.5=non OvOb and >0.5=OvOb). Individuals with a BMI<25.00 and median VAT rating of 8 have a median BMI value of 24.00; individuals with a BMI<25.00 and median VAT rating of 6 have a median BMI value of 23.50.

### DETAILED DESCRIPTION OF THE INVENTION

Overweight and obesity is a recognised pre-disease state. The current method of overweight and obesity (OvOb) classification using BMI and values of <25.00 to designate normal weight, 25.00 to ≤30.00 to designate overweight and >30.00 to designate obesity, is a methodology that does not address the underlying biochemistry of a condition that is a major risk factor of future disease including insulin resistance, diabetes and atherosclerosis, and in doing so, is failing to identify individuals of a pre-disease state. The invention relates to the identification of OvOb through biochemical measurements.

In a first aspect is a method of identifying an individual of BMI<25.00 with an overweight/obese biochemical phenotype comprising measuring the concentration of leptin or at least two biomarkers from an ex vivo sample obtained from the individual and based on the measurements establishing whether the individual has an overweight/obese biochemical phenotype. A possible explanation of ‘hidden obesity’ i.e. overweight/obese biochemical phenotypes in individuals with a BMI<25.00, could be that the individual has excessive and/or dysfunctional visceral adipose tissue. Some individuals of BMI ≥25.00 or ≥30.00 have a biochemical profile that does not indicate overweight/obesity which could be due to excess subcutaneous adipose tissue and would not necessarily represent a pre-diseased state such as insulin resistance and diabetes, although increased SAT could impact health in other ways. Nonetheless, a BMI of ≥25.00 is clinically acknowledged to be undesirable and to be a predictor of future health problems and the medical advice is to work towards a BMI of <25.00. Therefore, although the invention can be applied to individuals of any BMI, in a preferred embodiment it is applied to individuals whose BMI is <25.00 as these are the demographic who are currently not considered to be of a pre-diseased state and who are vulnerable to develop disease due to oversight.

The terms “subject”, “individual” and “patient” are used herein interchangeably to refer to the person whose biochemical phenotype is to be determined. The individual will typically have a body mass index (BMI) of less than 25.00. The BMI is a measurement of a person's leanness or corpulence based on height and weight, and is intended to quantify tissue mass. It is used widely as a general indicator of whether a person has a healthy body weight for their height. The BMI can be calculated by inputting height and weight in the following formula:

BMI=Weight (kg)+Height (m²).

The terms “biomarker” and “analyte are used interchangeably herein and refer to biological molecules present in a body which can be measured to obtain a concentration or other value which can be compared against a control value.

In the context of the present invention, a “control value” or simply “control” is understood to be the mean or median concentration of a particular biomarker/analyte typically found in healthy individuals of BMI<25.00 e.g. the median values for BMI<25.00 of Table 1; these mean or median control concentrations could further be defined by using boundaries such as ±1× the standard deviation mean value or ±the interquartile median concentration for the healthy, non-overweight/obese concentrations, and a value of a biomarker measured outside of the boundary would represent an undesirable state. It is appreciated that for an indication of an overweight/obese biochemical phenotype the concentration level of the biomarker would be on the side of the boundary signifying overweight/obese. The control value can also be the first biomarker measurement value of an individual who undergoes serial testing for the biomarker.

The present inventors have identified specific biomarkers that can be used to identify the overweight/obese phenotype. Various biomarkers were analysed using unpaired statistical comparisons of unmedicated, self-reported healthy individuals to establish their significance in determining the overweight/obese phenotype (see Table below).

TABLE 1
Median values of biochemicals in overweight/obese individuals of
BMI ≥25 versus individuals with BMI <25 and t-test or Mann-
Whitney statistic (P-value). Neither female nor male age distribution
were significantly different across BMI categories (<25 vs ≥25).

| | Females 23-75 years | | | Males 23-75 years | | |
|---|---|---|---|---|---|---|
| Biomarker | BMI <25 | BMI ≥25 | P-value | BMI <25 | BMI ≥25 | P-value |
| Complement CIII | 1.095 | 1.278 | <0.0001 | 1.047 | 1.286 | <0.0001 |
| Complement | 0.233 | 0.273 | 0.0002 | 0.231 | 0.277 | 0.0011 |
| CIV | | | | | | |
| C-peptide | 495 | 680 | <0.0001 | 503 | 670 | 0.0004 |
| Eosinophil | 0.11 | 0.13 | 0.0459 | 0.11 | 0.12 | 0.5331 |
| Ferritin | 40.50 | 41.00 | 0.5750 | 126 | 190 | 0.0072 |
| Glucose | 4.98 | 5.18 | 0.1709 | 5.33 | 5.47 | 0.1863 |
| Haemoglobin | 133 | 138 | 0.2986 | 151 | 153 | 0.1064 |
| HbA1c | 29.72 | 30.15 | 0.0639 | 30.52 | 30.80 | 0.6413 |
| HDL | 1.64 | 1.46 | 0.0912 | 1.50 | 1.15 | 0.0133 |
| hFABP | 2.12 | 2.44 | 0.2337 | 2.60 | 3.07 | 0.0346 |
| IgA | 2.15 | 2.095 | 0.9812 | 2.09 | 2.45 | 0.1046 |
| IgG | 10.82 | 10.13 | 0.4413 | 9.70 | 10.23 | 0.2317 |
| Insulin | 42.30 | 63.0 | <0.0001 | 34.7 | 64.5 | <0.0001 |
| Iron | 18.50 | 17.40 | 0.1627 | 19.2 | 21.3 | 0.3870 |
| LDL | 2.75 | 3.14 | 0.0020 | 3.53 | 4.23 | 0.0020 |
| Leptin | 5.06 | 18.07 | <0.0001 | 1.31 | 5.18 | <0.0001 |
| Monocyte count | 0.46 | 0.42 | 0.3169 | 0.45 | 0.49 | 0.3335 |
| Neutrophil count | 3.06 | 3.29 | 0.2880 | 2.63 | 2.82 | 0.6090 |
| Small LDL | 17.44 | 19.90 | 0.1528 | 21.41 | 31.43 | <0.0001 |
| TAS | 1.83 | 1.84 | 0.2745 | 1.87 | 1.95 | 0.0481 |
| TIBC | 63.35 | 62.60 | 0.7096 | 57.4 | 57.3 | 0.8584 |
| Total bilirubin | 11.68 | 9.86 | 0.0007 | 14.14 | 12.24 | 0.1401 |
| Total cholesterol | 5.03 | 5.41 | 0.1392 | 5.42 | 6.07 | 0.0212 |
| Transferrin | 2.76 | 2.67 | 0.5535 | 2.59 | 2.64 | 0.6946 |
| Triglycerides | 0.70 | 0.88 | 0.0026 | 0.75 | 1.29 | <0.0001 |
| Urea | 4.56 | 4.83 | 0.8510 | 5.14 | 5.42 | 0.0314 |
| Uric acid | 263 | 286 | 0.5239 | 327.2 | 393.5 | 0.0003 |
| hs-CRP | 0.55 | 1.49 | <0.0001 | 0.35 | 0.95 | <0.0001 |

From this analysis the following analytes were statistically significantly different at P<0.10:
List i. (females)-Complement component C3 (comp C3), Complement component C4 (comp C4), high sensitivity C-reactive protein (hs-CRP or CRP), eosinophil count, triglycerides, C-peptide, leptin, high density lipoprotein (HDL), low density lipoprotein (LDL), insulin, human glycated hemoglobin A1c (Hb1Ac) and total bilirubin
List ii. (males)-Comp C3, Comp C4, hs-CRP (or CRP), ferritin, hFABP, triglycerides, C-peptide, leptin, HDL, LDL, insulin, small LDL cholesterol, total cholesterol, TAS, urea, uric acid and total bilirubin

The measurement units of each of the above biomarkers (analytes) for any values disclosed herein are Comp C3 (g/l), Comp C4 (g/l), hs-CRP (mg/l), eosinophil count (10⁹/l), triglycerides (mmol/l), C-peptide (pmol/l), leptin (μg/l), HDL (mmol/l), LDL (mg/dl), insulin (pmol/l), Hb1Ac (mmol/mol), total bilirubin (μmol/l), ferritin (μg/l), hFABP (ng/ml), CRP (mg/l), small LDL cholesterol (mg/dl), total cholesterol (mmol/l), TAS (mmol/l), urea (mmol/l) and uric acid (μmol/l).

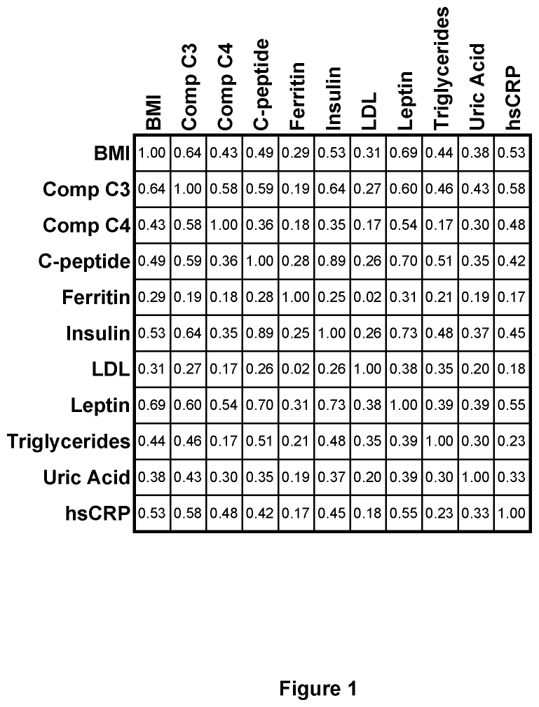

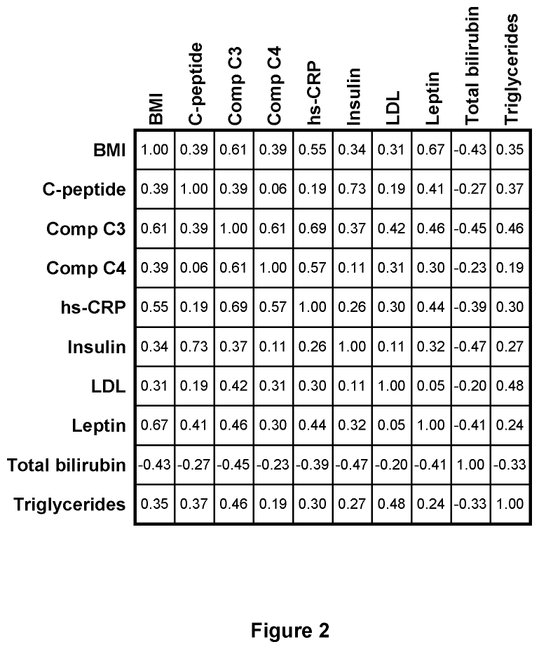

Each of the above analytes can therefore be used as an indicator of biochemical overweight/obesity either as a single biomarker or, more preferably, as a group of combined biomarkers. To consolidate the unpaired statistical analyses and support multiple variable analysis using the biomarkers (the ‘variables’) to discriminate overweight/obese phenotype and a non-overweight/non-obese phenotype a correlation analysis for each gender was implemented (see FIGS. 1 and 2).

When two or more biomarkers are to be used in the diagnostic method of identifying an overweight/obese biochemical phenotype, a suitable mathematical or machine learning classification model based on multiple variable analysis, such as a logistic regression equation, can be derived from existing biomarker measurements of individuals and the derived model used to categorise an individual of unknown biochemical phenotype. Such models as described herein may be referred to as “statistical methodologies”. The significance of the levels of the biomarkers, i.e. categorisation of the individual as either an OvOb biochemical phenotype or non-OvOb (non-overweight/non-obese) biochemical phenotype, can be established by inputting the biomarker concentration values into said model. Such a classification model may be chosen from one capable of analysing multiple variables (two or more variables), such as decision trees, K-nearest neighbour, artificial neural networks, regression methods such as logistic regression, random forests, principal component analysis, support vector machine or any other method of developing classification models known in the art. The output of the models used herein would indicate whether an individual, whether male or female, possesses an overweight/obese phenotype. Such an output could be a numerical value, for example a number between 0 and 1, an odds ratio value, a risk ratio/relative risk value or an alphabetic output such as ‘yes’ or ‘no’ or ‘high risk’, ‘low risk’ etc. One such statistical methodology is multiple logistic regression. As part of this methodology an individual, following analysis, is placed into one of two categories-is the individual of the overweight/obese biochemical phenotype or of the non-overweight/non-obese biochemical phenotype i.e. a ‘yes’ or ‘no’ output. A further method that can be used, which does not use a ‘named’ statistical methodology, involves highlighting individual biomarkers that have significantly different concentrations in overweight/obese populations (BMI≥25.00) vs non-overweight/non-obese populations (BMI<25.00) e.g. those highlighted in Table 1 using unpaired t-tests or Mann-Whitney statistic, which are then numerically classed as 1 or 0 depending on whether their measured concentration is more aligned to an overweight/obese phenotype or non-overweight/non-obese phenotype, respectively, and a score calculated based on combining two or more of the numbers to place them into one of the phenotypes. A further method that can be used is to track the concentration fluctuation of each individual biomarkers of Lists i. and ii. by taking and analysing samples from an individual with a BMI<25.00 at different time points Measuring biomarker concentrations in an individual at different time-points is also referred to as individual serial testing or measurement. If one or more of the biomarkers displays a concentration trend in the direction consistent with an overweight/obese phenotype over two or more consecutive time-points, this indicates an undesirable trend and preventative measures can be initiated to arrest or reverse the trend. In this method, the initial measured individual biomarker concentrations at time-point 1 can each be classed as a control value, to which ensuing measurements at time-points 2,3,4 etc, can be compared. The biomarkers of Lists i. and ii. would all show an increasing concentration trend in the transition from a non-overweight/obese biochemical phenotype to an overweight/obese biochemical phenotype, except for total bilirubin which would display a decrease. The different time points could be separated by weeks, months or years depending upon the analytical granularity required by the individual.

It is preferable to use a named multiple variable statistical methodology of the type mentioned previously for comparing measurement data to reference data. For example, the multiple logistic regression (MLR) equation for identifying a male overweight/obese biochemical phenotype vs a non-overweight/non-obese biochemical phenotype using the biomarker combination of Comp C3, leptin and triglycerides based on sample cohort described in the Methods and Results section is:

$$Y = - 7.751 + 6.134 [ Comp C 3 ] + 2.448 [ {log}_{10} leptin ] + 2.451 [ {log}_{10} triglycerides ]$$

whereby [biomarker] represents the biomarker concentration (AUC for this combination is 0.905-see Table 2). If Y is above zero for an individual of BMI<25.00 then the presence of an OvOb biochemical phenotype in the individual is supported, whereas if Y is below zero the presence of a non-OvOb biochemical phenotype is supported. The equation may also be used to indicate whether an individual with a BMI of ≥25.00 has a biochemical phenotype suggestive of non-OvOb; however, this utility is not as powerful as the methods of the invention as an individual who is overweight or obese is generally considered to be less healthy and more prone to disease. The equation was derived from data input into the statistics program Graphpad Prism 9.02. As the skilled person is aware, the biomarker coefficients of the equation will vary to a degree with a different sample cohort or if data from additional individuals is added to the model over time. Such changes would likely be observed whichever statistical methodology was applied. Although MLR is exemplified, use of similar-suited statistical methodologies does not affect the fundamental methodology of the invention which is the categorisation of the biochemical phenotype of an individual with a healthy BMI of <25.00 as healthy or unhealthy based on measurement of the biomarkers of the claims.

In a further embodiment of the invention, two or more different biomarker combinations are used to classify an individual as having an OvOb or non-OvOb biochemical phenotype. Either a single statistical methodology incorporating measurements from the two or more different biomarker combinations or two different statistical methodologies each incorporating a different biomarker combination can be used; the former approach is preferred. This approach has the advantage of maximising the identification of OvOb biochemical phenotypes in individuals with BMI<25.00 (or 24.00, 23.00 etc.) and mitigates against the possibility that a specific individual biomarker assay malfunctions thus compromising the statistical methodology in which it is incorporated. This approach is enabled due to the identification of several biomarkers, as presented in List i. (females) and List ii (males), being suitable for OvOb biochemical phenotyping. For example, a male individual of BMI 23.00 with biomarker measurement values that are within the reference range but whose values within this range are for leptin high, Comp C3 low, triglycerides high, C-peptide low, uric acid high, LDL high, ferritin, insulin high, Com C4 high, the biomarkers suggesting an OvOb biochemical phenotype, could be classed as OvOb using a statistical methodology (for example multiple logistic regression) which incorporates the biomarker concentrations of leptin, Comp C3 and triglycerides and non-OvOb using a statistical methodology which incorporates the biomarker concentrations of C-peptide, Comp C3 and triglycerides. The skilled person is aware that the variation applied to the statistical methodologies does not affect the fundamental methods described or the biomarkers used in the methods of the invention which are preferably combinations of two or more biomarkers taken from List i. (females) or List ii. (males) as described.

To establish whether the individual has an overweight/obese biochemical phenotype the biomarker measurements, taken from one or more ex vivo samples of the individual, may be compared to reference data. In the methods of the current invention phrases comprising wording ‘comparing biomarker measurements to reference data’, or words of the like, means comparing against normal control values/concentrations, preferably using a statistical methodology unless otherwise implied from the context. The preferred biomarkers to be used in the methods of the invention are chosen from two or more of complement C3 (Comp C3), leptin, triglycerides, high sensitivity C-reactive protein (hs-CRP) or C-reactive protein (CRP), C-peptide, LDL cholesterol (LDL) or small LDL cholesterol (small LDL), insulin, ferritin and complement C4 (Comp C4). Each of hs-CRP/CRP and LDL/small LDL are highly correlated and either of the two proteins in each of the pairs can be used in a biomarker combination.

It has been found that leptin on its own is statistically significant in determining the overweight/obese phenotype. Accordingly, in one embodiment, the measurement of leptin as a single biomarker may be used in the determination of the phenotype in individuals with a BMI of less than 25.00.

The biomarkers can be used to identify an overweight/obese biochemical phenotype (or a non-overweight/non-obese biochemical phenotype) in males or females. In addition, uric acid can also be used in combination with one or more of to the aforementioned biomarkers to identify a male overweight/obese biochemical phenotype and total bilirubin can also be used in combination with one or more of to the aforementioned biomarkers to identify a female overweight/obese biochemical phenotype. Preferred combinations of biomarkers are shown in Tables 2 and 3 for males and females, respectively. Table 2 Exemplary multiple logistic regression AUC statistic of leptin and also different biomarker combinations used to identify obesity & overweight in males using BMI as the dependent variable (N=65 BMI≥25, N=30 BMI<25)*

| Biomarkers | AUC |
|---|---|
| Leptin | 0.860 |
| Leptin + C-peptide | 0.874 |
| Leptin + small LDL | 0.866 |
| Leptin + insulin | 0.848 |
| Leptin + ferritin | 0.847 |
| Leptin + triglycerides | 0.884 |
| Leptin + hs-CRP | 0.859 |
| Leptin + HDL | 0.859 |
| Leptin + LDL | 0.861 |
| Leptin + Comp C4 | 0.869 |
| Leptin + Comp C3 | 0.897 |
| Leptin + uric acid | 0.868 |
| Comp C3 + uric acid | 0.838 |
| Comp C3 + ferritin | 0.871 |
| Comp C3 + LDL | 0.864 |
| Comp C3 + triglycerides | 0.880 |
| Comp C3 + Comp C4 | 0.830 |
| Comp C3 + insulin | 0.846 |
| Comp C3 + C-peptide | 0.839 |
| Comp C3 + hs-CRP | 0.831 |
| Comp C3 + insulin + leptin | 0.898 |
| C-peptide + LDL | 0.787 |
| C-peptide + triglycerides | 0.772 |
| Triglycerides + hs-CRP | 0.766 |
| Triglycerides + ferritin | 0.761 |
| Triglycerides + Comp C4 | 0.790 |
| Triglycerides + LDL | 0.768 |
| Triglycerides + insulin | 0.801 |
| hs-CRP + insulin | 0.780 |
| Insulin + Comp C4 | 0.788 |
| Insulin + LDL | 0.810 |
| Insulin + triglycerides + Comp C4 | 0.827 |
| Insulin + LDL + triglycerides | 0.837 |
| Leptin + Comp C3 + hs-CRP | 0.897 |
| Leptin + Comp C3 + triglycerides | 0.905 |
| Comp C3 + C-peptide + leptin | 0.900 |
| Comp C3 + ferritin + LDL | 0.875 |
| Comp C3 + small LDL + leptin | 0.901 |
| Comp C3 + triglycerides + leptin + insulin | 0.907 |
| Comp C3 + triglycerides + leptin + C-peptide | 0.907 |

* multiple logistic regression (MLR) applied to larger non-stratified dataset (includes medicated, diseased etc) for males aged 35-49 (N = 1,017) using the markers comp C3, leptin and LDL gave an AUC = 0.8125, thus adding support to the legitimacy of the analysis. LDL and small LDL are highly correlated and give similar results hence are interchangeable in the MLR & other statistical methodologies and both can be used in the methods of the invention: Pearson correlation r-value = 0.730 and multiple logistic regression for Comp C3 + small LDL + leptin gave an AUC = 0.901.

TABLE 3
Exemplary multiple logistic regression AUC statistic
of different biomarker combinations used to identify
obesity & overweight in females 23 to 75 years.

| Biomarkers | AUC |
|---|---|
| Leptin + C-peptide | 0.807 |
| Leptin + insulin | 0.803 |
| Leptin + ferritin | 0.869 |
| Leptin + triglycerides | 0.809 |
| Leptin + hs-CRP | 0.831 |
| Leptin + HDL | 0.842 |
| Leptin + LDL | 0.859 |
| Leptin + Comp C4 | 0.849 |
| Leptin + Comp C3 | 0.783 |
| Leptin + total bilirubin | 0.835 |
| Comp C3 + C-peptide | 0.817 |
| Comp C3 + hs-CRP | 0.800 |
| Comp C3 + Comp C4 | 0.739 |
| Comp C3 + insulin | 0.746 |
| Comp C3 + total bilirubin | 0.769 |
| Comp C3 + LDL | 0.735 |
| Comp C3 + triglycerides | 0.765 |
| Triglycerides + HDL | 0.752 |
| Triglycerides + hs-CRP | 0.777 |
| Triglycerides + insulin | 0.768 |
| Triglycerides + total bilirubin | 0.756 |
| Triglycerides + C-peptide | 0.773 |
| C-peptide + Comp C4 | 0.817 |
| C-peptide + ferritin | 0.760 |
| C-peptide + HDL | 0.807 |
| C-peptide + hs-CRP | 0.816 |
| C-peptide + LDL | 0.804 |
| LDL + hs-CRP | 0.800 |
| LDL + insulin | 0.739 |
| Insulin + hs-CRP | 0.790 |
| Leptin + insulin + total bilirubin | 0.844 |
| Comp C3 + C-peptide + hs-CRP | 0.861 |
| Comp C3 + C-peptide + total bilirubin | 0.852 |
| Leptin + LDL + triglycerides | 0.821 |
| C-peptide + LDL + hs-CRP | 0.860 |
| Comp C3 + C-peptide + hs-CRP + total bilirubin | 0.874 |

Preferred combinations for males include Comp C3+leptin, leptin+small LDL, leptin+Comp C3+small LDL, leptin+Comp C3+triglycerides and leptin+Comp C3+triglycerides+C-peptide; preferred combinations for females include Comp C3+leptin, Comp C3+C-peptide, Comp C3+C-peptide+hs-CRP and Comp C3+C-peptide+hs-CRP+total bilirubin.

In one embodiment any of the combinations in Tables 2 or 3 are used in the present invention. In a preferred embodiment, those combinations that have an indicated AUC value greater than 0.8, or more preferably 0.85, are used in the invention.

The use of Comp C3 and leptin as biomarkers of an OvOb biochemical phenotype in individuals of BMI<25.00 is particularly preferred; the combination of the biomarkers Comp C3 and leptin underpinning the methods of the invention either as a two-marker combination or together with further previously mentioned biomarkers has been found to be a powerful approach to the identification of hidden obesity. It has been found that overweight and obese biochemical phenotypes are also present in individuals of BMI<24.00, BMI<23.50 and BMI<22.50 and that the biomarkers previously described for use in the methods of the invention utilising a BMI value of <25.00 can indeed also be applied to methods using these BMI values of <22.50, <23.50 or <24.00 (see Tables 4 to 9); BMI values of anywhere between <22.50 to <25.00 are suitable for use in the methods of the invention and include a BMI value of <24.90, <24.80, <24.70, <24.60, <24.50, <24.40, <24.30, <24.20, <24.10, <24.00, <23.90, <23.80, <23.70, <23.60, <23.50, <23.40, <23.30, <23.20, <23.10, <23.00, <22.90, <22.80, <22.70, <22.60 and <22.50. It is possible that a BMI of less than between 22.50 and about 22.00 would also possess discriminatory diagnostic power. This discovery has potential ramifications for the BMI standard of <25.00 to denote a person of healthy height to weight ratio and could support improved preventative healthcare processes by identifying individuals at greater risk of diseases such as insulin resistance, diabetes and atherosclerosis who have previously been categorised as being healthy and not at risk based on a BMI of <25.00. Accordingly, in one embodiment, the individual will have a BMI of from 22.00 to 25.00, more preferably from 23.00 to 25.00.

TABLE 4
T-test and MW statistics of biomarker for BMI <24.00
vs BMI of 24.00 to <26.00 in males (N = from
18 to 25 dependent upon biomarker)

| | Median | | |
|---|---|---|---|
| | | BMI 24.00 to | |
| Biomarker | BMI <24.00 | <26.00 | P value |
| Comp C3 | 1.055 | 1.155 | 0.0449 |
| Leptin | 1.430 | 2.285 | 0.0605 |
| LDL | 21.87 | 27.36 | 0.182 |
| Triglycerides | 0.680 | 0.865 | 0.102 |
| Uric acid | 332 | 379 | 0.0681 |

TABLE 5
Multiple logistic regression AUC statistic of
different biomarker combinations for BMI <24.00
vs BMI of 24.00 to <26.00 in males

| Biomarkers | AUC |
|---|---|
| Comp C3 + LDL + leptin | 0.902 |
| Comp C3 + LDL + leptin + triglycerides | 0.774 |
| Comp C3 + LDL + leptin + triglycerides + uric acid | 0.778 |
| Comp C3 + LDL + leptin + triglycerides + hs-CRP | 0.791 |

TABLE 6
T-test and MW statistics of biomarker for BMI ≤23.50
vs BMI of >23.50 to <26.00 in males

| | Median | | |
|---|---|---|---|
| | | BMI 23.50 to | |
| Biomarker | BMI <23.50 | <26.00 | P value |
| Comp C3 | 1.046 | 1.159 | 0.0203 |
| Leptin | 1.420 | 2.410 | 0.0383 |
| LDL | 22.96 | 27.27 | 0.5231 |
| Triglycerides | 0.680 | 0.865 | 0.0846 |
| Uric acid | 343 | 369 | 0.2089 |

TABLE 7
Multiple logistic regression AUC statistic of
different biomarker combinations for BMI ≤23.50
vs BMI of >23.50 to <26.00 in males

| Biomarkers | AUC |
|---|---|
| Comp C3 + LDL + leptin | 0.727 |
| Comp C3 + LDL + leptin + triglycerides | 0.850 |
| Comp C3 + LDL + leptin + triglycerides + uric acid | 0.850 |
| Comp C3 + LDL + leptin + triglycerides + hs-CRP | 0.788 |

TABLE 8
T-test and MW statistics of biomarker for BMI ≤22.50
vs BMI of >22.50 to <26.00 in females

| | Median | | |
|---|---|---|---|
| | | BMI 22.50 to | |
| Biomarker | BMI <22.50 | <26.00 | P value |
| Comp C3 | 1.053 | 1.175 | 0.0010 |
| C-peptide | 475 | 577 | 0.0128 |
| Leptin | 4.58 | 11.93 | 0.0008 |
| LDL | 2.595 | 3.130 | 0.0196 |
| Triglycerides | 0.675 | 0.890 | 0.0174 |
| hs-CRP | 0.535 | 0.840 | 0.0113 |
| Total bilirubin | 12.24 | 10.28 | 0.0598 |

TABLE 9
Multiple logistic regression AUC statistic of different
biomarker combinations for BMI ≤22.50 (N =
40) vs BMI of >22.50 to <26.00 (N = 36)
in females (Leptin undermines attempted analysis
due to substantially fewer data points)

| Biomarkers | AUC |
|---|---|
| Comp C3 + C-peptide + hs-CRP | 0.767 |
| Leptin + LDL + triglycerides | 0.775 |
| C-peptide + LDL + hs-CRP | 0.736 |
| Comp C3 + C-peptide + hs-CRP + total bilirubin | 0.762 |

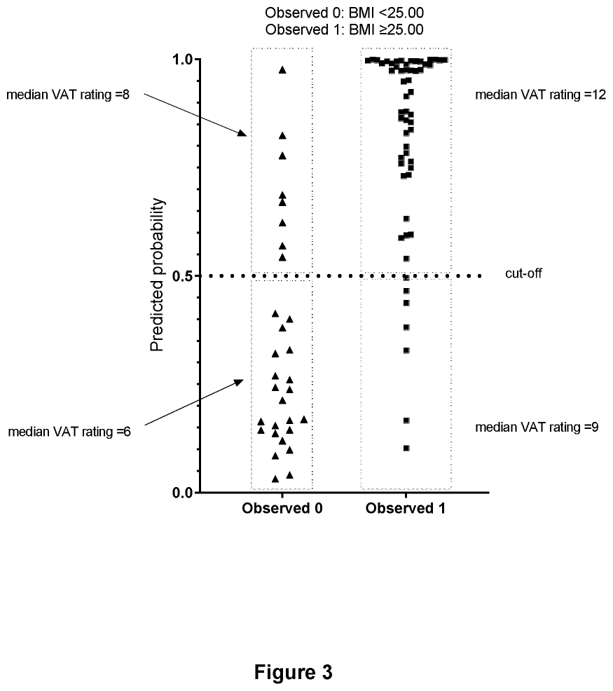

It will be recognised that the concept described herein can equally be applied to identifying individuals with a BMI≥25.00 who have a non-overweight/non-obese biochemical phenotype. Hence in a further aspect of the invention is a method of identifying an individual of BMI≥25.00 who has a non-overweight/non-obese biochemical phenotype (a healthy biochemical phenotype) comprising measuring the concentration of at least two biomarkers from an ex vivo sample obtained from the individual and based on the measurements establishing whether the individual has a non-overweight/non-obese biochemical phenotype. Hence the methods and kits of the invention may also be used to ‘rule out’ an OvOb biochemical phenotype in an individual with a BMI ≥25.00. Referral to FIG. 3 shows that data points <0.5 for predicted probability for individuals in the observed 1 category qualify for a non-OvOb (non-overweight/non-obese) biochemical phenotype.

The sample (biological sample) obtained from a patient prior to its analysis is preferably a blood, serum, plasma or urine sample.

As used herein, the term ‘ex vivo’ has its usual meaning in the art and refers to a sample that has been removed from a patient's body. When a blood sample is taken from the patient for analysis, whole blood, serum or plasma is analysed. Analysis of the blood sample can be by way of several analytical methodologies such as mass spectrometry optionally inked to a pre-separation step such as chromatography or preferably an enzymatic assay, an agglutination assay or an immunoassay using a clinical analyser.

The biomarkers/analytes may be analysed using conventional techniques and standard assays established for the biomarkers/analytes. When combinations of biomarkers are to be measured, it may be beneficial to obtain more than one sample from the patient. However, typically, all of the biomarkers can be measured from a single sample, preferably a blood sample.

Different assays may be performed depending on which biomarker is being determined. Commercially available kits or assays may be used to determine the concentration levels in a patient sample. The following commercially available kits/assays may be used:
Complement C3: manufactured by Randox Laboratories, Catalogue no. CM 3845, for use with RX Series analyzers (Daytona/Imola) using serum or plasma;
Complement C4: manufactured by Randox Laboratories, Catalogue no. CM 3845, for use with RX Series analyzers (Daytona/Imola) using serum or plasma;
C Reactive Protein: manufactured by Randox Laboratories, Catalogue no. 3847, for use with RX Series analyzers (Daytona/Imola) using serum or plasma;
Ferritin: manufactured by Randox Laboratories, Catalogue no. FN 3888, for use with RX Series analyzers (Daytona/Imola) using serum or plasma;
Insulin (Elecsys Insulin Assay): manufactured by Roche Diagnostics, Application code no. 10059, for use with Cobas 801 analyzer using serum or plasma;
C-peptide (Elecsys C-peptide Assay): manufactured by Roche Diagnostics, Application code no. 10081, for use with Cobas 801 analyzer using serum, plasma or urine;
Triglycerides: manufactured by Randox Laboratories, Catalogue no. 3823, for use with RX Series analyzers (Daytona/Imola) using serum or plasma;
Total bilirubin: manufactured by Randox Laboratories, Catalogue no. BR 8377, for use with RX Daytona+ using serum or plasma;
LDL: manufactured by Randox Laboratories, Catalogue no. CH 8032, for use with several clinical analyzers including RX Modena, Cobas Mira & Hitachi 717, using serum or plasma;
Small LDL: manufactured by Randox Laboratories, sLDL-EX “Seiken” Catalogue no. 562616, for use with Roche/Hitachi 717, using serum or plasma;
Uric acid: manufactured by Randox Laboratories, Catalogue no. CH 3824, for use with RX series analyzers (Daytona/Imola), using serum, plasma or urine;
hs-CRP (high sensitivity C-reactive protein): manufactured by Roche Diagnostics, Application code nos. 217/8217, for use with Cobas 311,501,502 analyzers using serum or plasma;
Leptin: manufactured by Randox Laboratories, Catalogue no. EV 4219 (Biochip Metabolic Syndrome Array), for use with Evidence series analyzers (Evidence Evolution) using serum.

Other kits/assays are available as will be understood by the skilled person.

A preferred methodology is based on immuno-detection (an assay incorporating antibodies). Immuno-detection technology is also readily incorporated into transportable or hand-held devices for use outside of the clinical environment. A quantitative immunoassay such as a Western blot or ELISA can be used to detect the amount of protein. A preferred method of analysis comprises using a multi-analyte biochip which enables several proteins to be detected and quantified simultaneously. A biochip is a planar substrate that may be, for example, mineral or polymer based, but is preferably ceramic. Probes are adsorbed on or chemically attached to the surface of the biochip. The probes can be any biomarker-specific probe or binding ligand to the OvOb protein biomarkers mentioned. As used herein, the term ‘specific’ means that the probe binds only to one of the biomarkers of the methods and kits of the invention, with negligible binding to other biomarkers of the invention or to other analytes in the biological sample being analysed. This ensures that the integrity of the diagnostic assay and its result using the biomarkers of the invention is not compromised by additional binding events. When identifying the various biomarkers of the invention it will be apparent to the skilled person that for protein biomarkers as well as identifying the full-length protein, the identification of a fragment or several fragments of a protein is possible, provided this allows accurate identification of the protein. Similarly, although a preferred probe of the invention is a polyclonal or monoclonal antibody, other probes such as aptamers, molecular imprinted polymers, phages, short chain antibody fragments, single domain antibodies and other antibody-based probes may be used. A solid-state device can be used in the methods of the present invention to support the binding ligands such as a microtitre plate, polymer-based beads and plastic, glass or ceramic chips (also referred to as ‘biochips’).

In a further aspect of the invention there is described a diagnostic kit dedicated to the identification of an overweight/obese biochemical phenotype. The diagnostic test kit incorporates binding ligands each specific to a single biomarker, the biomarkers being complement C3 and/or leptin, and, if only one of complement C3 or leptin is present one or more of C-peptide, CRP, triglycerides, small LDL, LDL, complement C4, ferritin, total bilirubin, uric acid and insulin; if both complement C3 and leptin binding ligands are present the kit optionally contains one or more of C-peptide, CRP, triglycerides, small LDL, LDL, complement C4, ferritin, uric acid, total bilirubin and insulin. In a preferred embodiment the binding ligands are adsorbed or chemically attached (covalently bonded) to the surface of a biochip.

The binding ligand(s) is preferably an antibody, the definition of which includes antibody fragments, as will be understood by the skilled person.

In a further aspect of the invention, there is an assay comprising the steps of:
conducting on a sample of blood, plasma, serum or urine from a subject an analysis of leptin or analysis of any of the biomarker combinations defined in the first aspect of the invention or in any of Tables 2 or 3, to thereby
measure the concentration of said biomarker(s) from the sample, and providing a measurement of the concentration of said biomarker(s),
wherein said subject has a BMI of less than 25.00, and
wherein an increase in the measured concentration of said biomarker(s) compared to a control sample or value is indicative of overweight/obese phenotype.

In a further aspect of the invention, there is a method of detecting biomarkers of an overweight/obese phenotype in a patient having a BMI of less than 25.00, comprising
measuring the level of leptin or two or more of the biomarkers as defined in the first aspect of the invention or in any of Tables 2 or 3, in a sample or samples obtained from said patient, and
determining whether the measured level for each biomarker exceeds a control value for each biomarker.

The invention will now be described with reference to the accompanying figure in the following non-limiting examples.

### Methods and Results

Patients The individuals taking part in the study were self-reported healthy individuals, not taking medication and with no known morbidities. The statistical analyses were age (23 to 75 years) and gender stratified. Populations of healthy males and females with BMI<25.00 (normal/under-weight) versus ≥25.00 (overweight & obese) were initially compared using the Mann-Whitney statistic. All cohorts in the study were individuals not on medication and who reported a disease-free status.

Collection and Analysis of In Vitro Samples Each patient attended a Randox Health clinic and urine and blood samples were collected from the patient, a physical examination conducted and a health report form completed. The physical examination includes measuring weight, height, blood pressure, body fat measurements, reflex measurements and reflex tests. The Tanita MC-980 Body Composition Analyzer (Tanita Corporation, Japan) which is based upon electrical impedance supported various physiological measurements including a visceral fat rating which provides an indication of visceral fat level, the higher the rating indicative of a greater amount of visceral fat. Once the patient sample was collected, it was analyzed on-site or transferred to a Randox Laboratory for analysis. The samples were processed and analysed using Randox clinical analysers and other analysers from various manufacturers. Biochemicals were analysed at Randox Health Holywood, Randox Health London, Randox Health Liverpool and Randox Clinical Laboratory Services in Antrim. Analysers used for measuring biochemicals were Randox Imola/Monza/Daytona/Modena Randox Evolution, Sysmex XS1000i, Roche e801, Roche Cobas Mira, Roche Cobas 311/501/502, Hitachi 717, Roche Urisys 1100 and Siemens Immulite 2000XPi. Each analyser is used in its standard manner without modification and the analytes measured as per the accompanying instruction sheet/information for use (IFU).

Statistical Analysis Population data: two-tail unpaired t-test, with data transformation where appropriate for normalisation, and Mann-Whitney statistics were used for comparison of the population data; correlation analysis of any statistically significant biomarkers together with BMI was also computed using an upper threshold of significance of P<0.10. These results helped inform multiple logistic regression analyses of various biomarker measurement combinations using BMI values for overweight and obesity of ≥25.00, ≥24.00 or ≥23.50 versus the less than values for non-overweight and non-obese as the dependent variables (see Tables for biomarker combinations and results). A default cut-off value of 0.5 was used to derive the ROC curve and AUC values for the multiple logistic regression model for the various biomarker combinations. All calculations were effected using Graphpad Prism 9.02 software.

TABLE 1
Median values of biochemicals in overweight/obese individuals of
BMI ≥25 versus individuals with BMI <25 and t-test or Mann-
Whitney statistic (P-value). Neither female nor male age distribution
were significantly different across BMI categories (<25 vs ≥25).

| | Females 23-75 years | | | Males 23-75 years | | |
|---|---|---|---|---|---|---|
| Biomarker | BMI <25 | BMI ≥25 | P-value | BMI <25 | BMI ≥25 | P-value |
| Complement CIII | 1.095 | 1.278 | <0.0001 | 1.047 | 1.286 | <0.0001 |
| Complement | 0.233 | 0.273 | 0.0002 | 0.231 | 0.277 | 0.0011 |
| CIV | | | | | | |
| C-peptide | 495 | 680 | <0.0001 | 503 | 670 | 0.0004 |
| Eosinophil | 0.11 | 0.13 | 0.0459 | 0.11 | 0.12 | 0.5331 |
| Ferritin | 40.50 | 41.00 | 0.5750 | 126 | 190 | 0.0072 |
| Glucose | 4.98 | 5.18 | 0.1709 | 5.33 | 5.47 | 0.1863 |
| Haemoglobin | 133 | 138 | 0.2986 | 151 | 153 | 0.1064 |
| HbA1c | 29.72 | 30.15 | 0.0639 | 30.52 | 30.80 | 0.6413 |
| HDL | 1.64 | 1.46 | 0.0912 | 1.50 | 1.15 | 0.0133 |
| hFABP | 2.12 | 2.44 | 0.2337 | 2.60 | 3.07 | 0.0346 |
| IgA | 2.15 | 2.095 | 0.9812 | 2.09 | 2.45 | 0.1046 |
| IgG | 10.82 | 10.13 | 0.4413 | 9.70 | 10.23 | 0.2317 |
| Insulin | 42.30 | 63.0 | <0.0001 | 34.7 | 64.5 | <0.0001 |
| Iron | 18.50 | 17.40 | 0.1627 | 19.2 | 21.3 | 0.3870 |
| LDL | 2.75 | 3.14 | 0.0020 | 3.53 | 4.23 | 0.0020 |
| Leptin | 5.06 | 18.07 | <0.0001 | 1.31 | 5.18 | <0.0001 |
| Monocyte count | 0.46 | 0.42 | 0.3169 | 0.45 | 0.49 | 0.3335 |
| Neutrophil count | 3.06 | 3.29 | 0.2880 | 2.63 | 2.82 | 0.6090 |
| Small LDL | 17.44 | 19.90 | 0.1528 | 21.41 | 31.43 | <0.0001 |
| TAS | 1.83 | 1.84 | 0.2745 | 1.87 | 1.95 | 0.0481 |
| TIBC | 63.35 | 62.60 | 0.7096 | 57.4 | 57.3 | 0.8584 |
| Total bilirubin | 11.68 | 9.86 | 0.0007 | 14.14 | 12.24 | 0.1401 |
| Total cholesterol | 5.03 | 5.41 | 0.1392 | 5.42 | 6.07 | 0.0212 |
| Transferrin | 2.76 | 2.67 | 0.5535 | 2.59 | 2.64 | 0.6946 |
| Triglycerides | 0.70 | 0.88 | 0.0026 | 0.75 | 1.29 | <0.0001 |
| Urea | 4.56 | 4.83 | 0.8510 | 5.14 | 5.42 | 0.0314 |
| Uric acid | 263 | 286 | 0.5239 | 327.2 | 393.5 | 0.0003 |
| hs-CRP | 0.55 | 1.49 | <0.0001 | 0.35 | 0.95 | <0.0001 |

Tables 4 to 9 highlight that further BMI values can be used in the methods of the invention. This suggests that the onset to disruption of metabolic homeostasis, possibly due to adipocyte proliferation/dysregulation, is occurring in individuals with BMI values as low as about 22.50. The above data was extracted from individuals with no underlying health conditions and who were not taking medication; analysis of a larger dataset of males (N=300) and females (N=200) who were not controlled for medication taking or underlying health conditions produced similar results further confirming that the biomarkers and methods of the invention can be used for the identification of overweight and obese biochemical phenotypes in males and females with a BMI of less than 25.00. Although the methods of the invention can be successfully applied to identify an overweight/obese biochemical phenotype in individuals of BMI<25.00 without age stratification (the current methods used individuals aged 23 to 75 years), and incorporating age as an additional variable into the statistical methodologies had minimal impact on the output statistics (see Table 10a), there are slight BMI/biomarker concentration changes with age within BMI cohorts of <25.00 vs ≥25.00 (See Table 10b), and age stratification may be incorporated in the statistical methodology to tweak the diagnostic accuracy further. The visceral fat rating statistics support the possibility that the varied biochemical phenotypes could relate to visceral fat deposits (see FIG. 3).

TABLE 10a
Comparison of diagnostic power using multiple logistic
regression w/wo age as an additional variable

| Biomarkers | AUC |
|---|---|
| HB biomarkers: comp C3 + leptin + triglycerides | 0.905 |
| HB biomarkers: comp C3 + leptin + triglycerides + Age | 0.907 |
| (23-75 yrs) | |

TABLE 10b
Median values of BMI and biomarkers in different male age
cohorts for BMI ≥25.00 and BMI <25.00. This data
was derived from a large dataset of individuals of varying
health status (N = between 500 and 1,700 for each measurand).

| | BMI ≥25.00 | | BMI <25.00 | |
|---|---|---|---|---|
| | Age 35-49 | Age 25-34 | Age 35-49 | Age 25-34 |
| Measurand | yrs | yrs | yrs | yrs |
| BMI median | 28.20 | 27.40 | 23.60 | 22.80 |
| Leptin median | 4.90 | 4.54 | 1.45 | 1.33 |
| hs-CRP median | 1.03 | 1.14 | 0.585 | 0.530 |
| Triglycerides | 1.25 | 1.10 | 0.89 | 0.81 |
| median | | | | |

1. A method of identifying an individual with an overweight/obese biochemical phenotype whose BMI is less than 25.00 comprising measuring the concentration of the biomarker leptin, or at least two biomarkers from an ex vivo sample or samples obtained from the individual and based on the measurements establishing whether the individual has an overweight/obese biochemical phenotype, wherein the at least two biomarkers are chosen from complement C3, leptin, small LDL, triglycerides, hs-CRP, C-peptide, LDL, ferritin, insulin and complement C4.

2. The method of claim 1 which comprises the biomarkers leptin and complement C3 and/or small LDL.

3. The method of claim 1 in which the individual's BMI is less than 24.50, less than 24.00, less than 23.50, less than 23.00, or less than 22.50.

4. The method of claim 1 in which the establishment of whether the individual with BMI<25.00 has an overweight/obese biochemical phenotype is effected by inputting the biomarker measurements into a statistical methodology which categorizes individuals as non-overweight/obese or overweight/obese based on their biomarker measurements.

5. The method of claim 4 in which the biomarker measurements of at least two different biomarker combinations are each input into separate statistical methodologies.

6. The method of claim 5 in which the statistical methodology incorporates gender and/or age stratification.

7. The method of claim 6 which also includes the biomarker uric acid if the individual is male, or the biomarker total bilirubin if the individual is female.

8. The method of claim 1, wherein the at least two biomarkers are any of the combinations set out in Table 2a for males or Table 3 for females.

9. The method according to claim 8, wherein the combinations are any of those in Table 2a or Table 3 with an indicated AUC value greater than 0.8.

10. An assay comprising the steps of:
conducting on a sample of blood, plasma, serum or urine from a subject an analysis of leptin or an analysis of any of the biomarker combinations defined in any of one of claims 1, 7, 8 or 9, to thereby
measure the concentration of said biomarker(s) from the sample, and providing a measurement of the concentration of said biomarker(s),
wherein said subject has a BMI of less than 25.00, and
wherein an increase in the measured concentration of said biomarker(s) compared to a control sample or value is indicative of overweight/obese phenotype.

11. A method of detecting biomarkers of an overweight/obese phenotype in a patient having a BMI of less than 25.00, comprising
measuring the level of leptin or two or more of the biomarkers as defined in any of one of claims 1, 7, 8 or 9, in a sample or samples obtained from said patient, and
determining whether the measured level for each biomarker exceeds a control value for each biomarker.

12. A kit for use in the diagnosis of an overweight/obese biochemical phenotype in a subject comprising analyte-specific probes for at least two of the biomarkers defined in any of one of claims 1, 7, 8 or 9.

13. A diagnostic test kit incorporating at least two different binding ligands, each specific to a single biomarker, the at least two biomarkers comprising complement C3 and leptin.

14. The diagnostic kit of claim 13 in which the binding ligands are antibodies.

15. The diagnostic test kit of claim 14 in which the antibodies are adsorbed or attached to the surface of a biochip.

16. The diagnostic kit of claim 13, wherein the biomarkers are any combination of uric acid, total bilirubin, any two of the biomarkers in Table 2a or Table 3.

17. The use of leptin or leptin and complement C3 and/or small LDL as biomarkers of an individual with an overweight or obese biochemical phenotype.

18. The use of claim 17 in which the individual has a BMI of less than 25.00.

19. The use of claim 18 in which the individual's BMI is less than 24.50.

20. The use of claim 17 which further incorporates one or more biomarkers chosen from triglycerides, C-peptide, hs-CRP,LDL, complement C4, ferritin, insulin, total bilirubin and uric acid.
